(19) United States
(12) Patent Application Publication
(10) Pub. No.: US 2018/0092944 A1
(43) Pub. Date: Apr. 5, 2018

(54) ACTIVATED CHARCOAL PATCH

(71) Applicant: Neobiotech Global Corporation, Auburn, WA (US)

(72) Inventors: Joon Hyun Kim, Auburn, WA (US); Esther M. Park Hwang, Tacoma, WA (US); Gyeong Ye Choi, Auburn, WA (US)

(73) Assignee: Neobiotech Global Corporation

(21) Appl. No.: 15/283,637

(22) Filed: Oct. 3, 2016

(51) Int. Cl. A61K 33/44; A61K 9/70; A61K 9/00; A61K 45/06

(52) U.S. Cl. CPC A61K 33/44; A61K 45/06; A61K 9/0014; A61K 9/703

(57) ABSTRACT

This invention relates to an activated charcoal patch, which can be applied on a user's skin. The activated charcoal patch includes a hydrogel with activated charcoal particles dispersed therein. Upon applying the activated charcoal patch, the activated charcoal particles in the hydrogel absorb or extract the pathogens, toxic chemicals, inflammatory mediators and other undesired radicals from the skin. Also included within the scope of the invention are bioactive agents, such as insect repellants, sun screens, cosmetic agents, etc.

### FIELD OF THE INVENTION

The present invention relates to activated charcoal patch, in particular, activated charcoal patch suitable for topical application on skin.

### BACKGROUND OF THE INVENTION

It has long been known that charcoal may be rendered highly adsorptive by heating in a suitable atmosphere, the product of such treatment commonly being referred to as “activated charcoal”. Many applications have been found for the adsorptive properties of activated charcoal, particularly in the adsorption of undesirable contaminants from gases and liquids. Such activated charcoal has been found to have a particular utility in skin irritation soothing purposes, where it has been found to adsorb bacteria, wound exudate and undesired toxins from skin.

Nevertheless, cosmetic and medical uses of activated charcoal have been limited by the form which the activated charcoal is produced. For instance, activated charcoal is generally produced in a form of small pellets or powder as its production processes results in a product which was extremely friable and difficult to handle in any other form. To use activated charcoal for medical and cosmetic purposes, attempts have been made to incorporate an activated charcoal layer in transdermal drug-in-adhesive patch.

Problems encountered with such activated charcoal patches have included inefficient control over the release timing of the transdermal drugs and activated charcoal. In some previously known drug-in-adhesive patch, the activated carbon layer has been arranged between an antimicrobial agent layer and a cover layer. In this configuration, the layer containing the activated carbon is already covered by the antimicrobial agent layer, so that it is no longer fully available for the adsorption of malodorous substances, bacteria, or the like. In some other previously known adhesive patch with activated charcoal, the patch was provided with a layer that contains activated charcoal but without antimicrobial agent layer. In such cases, the activated carbon layer was disposed between a permeable material layer and another layer made of a semipermeable material of which the permeable layer would be positioned towards the skin when applying the patch. The activated charcoal layer interposed between the two layers could provide some degree of antibacterial properties, because the growth of the bacteria adsorbed on the layer containing the activated charcoal is inhibited by restriction of the oxygen supply. However, a problem here is that the physiological conditions for acceleration of wound healing are significantly worsened by the limited air permeability.

As such, further improvements are sought in the field of activated charcoal patches.

### SUMMARY OF THE INVENTION

This invention relates to activated charcoal patch for the delivery of active materials such as a drug through the skin.

The activated charcoal patch includes a hydrogel, which contains activated charcoal particles dispersed therein. The hydrogel is applied to an impervious backing sheet in which the hydrogel comprises activated charcoal combined with one or more host materials so that the hydrogel maintains a solid gel-like form on the impervious backing sheet.

In some embodiments, the hydrogel is preferably present in the form of a multi-layer sheet, each portion constituting a layer. In an embodiment, the hydrogel disposed on the backing sheet comprises an inner hydrogel sub-layer and an outer hydrogel sub-layer. The outer hydrogel sub-layer, which is positioned away from the backing sheet (i.e., positioned towards the skin upon applying the patch) may comprises activated charcoal dispersed therein. The inner hydrogel sub-layer, which is interposed between the outer hydrogel sub-layer and the backing sheet, may comprise active materials. In this configuration, activation of the outer hydrogel sub-layer occurs prior to the activation of the inner hydrogel sub-layer. Thus, the activated charcoal particles provided in the outer hydrogel sub-layer is released first to absorb undesired toxins, bacteria, fungus, carcinogens and other harmful pathogens, and the activation of the inner hydrogel sub-layer follows thereafter to release the active materials comprised in the inner hydrogel sub-layer. The active materials of the inner hydrogel sub-layer may include a transdermal pain relief drug.

The first active material and the second active material may be selected from the group consisting of hypnotics, sedatives, tranquilizers, anti-convulsants, muscle relaxants, analgesics, antipyretic agents, anti-inflammatory agents, local anesthetics, antispasmodics, anti-ulcer agents, anti-virals, anti-bacterials, anti-fungals, sympathomimetic agents, cardiovascular agents and antitumor agents.

In cases where the treatment by the activated charcoal itself would cause discomforts or pain to the patient, the transdermal drug may be provided in the outer hydrogel sub-layer and the activated charcoal particles may be provided in the inner hydrogel sub-layer. In this way, the transdermal pain relief drug in the outer hydrogel sub-layer is released first to anaesthetize the application area, then the inner hydrogel sub-layer is activated after the outer hydrogel sub-layer to release the activated charcoal particles to absorb undesired toxins, bacteria, fungus, carcinogens and other harmful pathogens. Because the pain relief drug of the outer hydrogel sub-layer was released beforehand, the activated charcoal particles can act on the application area with lesser degree of discomforts to the user.

Each of the outer hydrogel sub-layer and the inner hydrogel sub-layer may be formed of natural hydrogel polymer or hydrogel synthetic polymer. In some embodiments, each of the outer hydrogel sub-layer and the inner hydrogel sub-layer may be formed of at least one of an acrylic-based hydrogel, a polyisobutylene hydrogel and a silicone-based hydrogel.

In some embodiments, the outer hydrogel sub-layer has a first swelling activation trigger, and the inner hydrogel sub-layer has a second swelling activation trigger. In other words, the outer hydrogel sub-layer and the inner hydrogel sub-layer start to swell under different conditions, and allow the activated charcoal particles to serve their purposes and/or to release the active materials included in the hydrogel layer.

In an embodiment, the hydrogel is formed of a plurality of thermo-responsive hydrogel sub-layers, including at least one hydrogel sub-layer having a first lower critical solution temperature and at least one hydrogel sub-layer having a second lower critical solution temperature different from the first lower critical solution temperature.

In another embodiment, the hydrogel is formed of a plurality of ph-responsive hydrogel sub-layers, including at least one hydrogel sub-layer responsive to a first pH-level, and at least one other hydrogel sub-layer responsive to a second pH-level.

In an embodiment, at least one of the hydrogel sub-layers is a thermos-responsive hydrogel, and at least another one of the hydrogel sub-layers is a pH-responsive hydrogel sub-layer. For example, the swelling activation trigger for one of the hydrogel layers may be pH change, while the swelling activation trigger for another one of the hydrogel layers is temperature change across a predetermined threshold temperature.

In some embodiments, the hydrogel of the activated charcoal patch further includes an intermediate hydrogel sub-layer interposed between an inner hydrogel sub-layer and an outer hydrogel sub-layer. In one embodiment, the inner hydrogel sub-layer and the outer hydrogel sub-layer, including at least one common active material therein, may have different swelling activation trigger. In other words, at least one active material is provided in both the inner and outer hydrogel sub-layers, even though the inner hydrogel sub-layer has a first swelling activation trigger and the outer hydrogel sub-layer has a second swelling activation trigger. The activated charcoal particles may be provided in at least one of the inner hydrogel sub-layer, the intermediate hydrogel sub-layer and the outer hydrogel sub-layer.

The intermediate hydrogel sub-layer interposed between the inner hydrogel sub-layer and the outer hydrogel sub-layer may have a third swelling activation trigger. The swelling activation trigger of the intermediate hydrogel sub-layer may be the same as that of either the inner hydrogel sub-layer or the outer hydrogel sub-layer. Alternatively, the swelling activation trigger of the intermediate hydrogel sub-layer may be different from the swelling activation triggers of the inner hydrogel sub-layer and the outer hydrogel sub-layer. The active material commonly present in both the inner hydrogel sub-layer and the outer hydrogel sub-layer is not present in the intermediate hydrogel sub-layer.

Additional aspects related to these embodiments will be set forth in part in the description which follows, and in part will be apparent from the description or may be learned by practice of the invention. Aspects of the invention may be realized and attained by means of the elements and combinations of various elements and aspects particularly pointed out in the following detailed description and the appended claims.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention.

FIG. 1 is a cross-sectional view schematically illustrating an activated charcoal patch according to an exemplary embodiment;

FIG. 2 is a cross-sectional view schematically illustrating a transdermal patch according to an exemplary embodiment;

FIG. 3 is a cross-sectional view schematically illustrating an activated charcoal patch according to an exemplary embodiment of the disclosure.

### DETAILED DESCRIPTION OF THE INVENTION

In the following detailed description, reference will be made to the accompanying drawings. The aforementioned accompanying drawings show by way of illustration, and not by way of limitation, specific embodiments and implementations consistent with the principles of the present invention. These implementations are described in sufficient detail to enable those skilled in the art to practice the invention, and it is to be understood that other implementations may be utilized and that structural changes and/or substitutions of various elements may be made without departing from the scope and spirit of the invention.

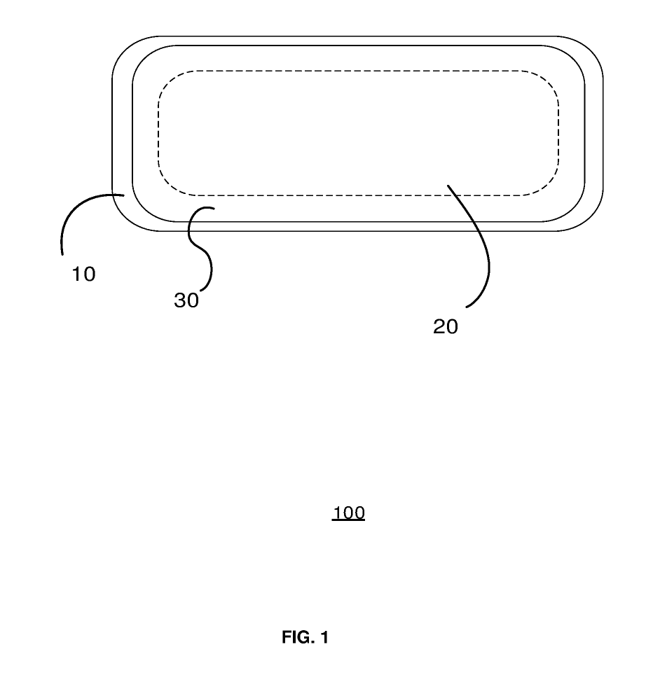

FIG. 1 is a top view of an embodiment of activated charcoal patch, Referring to FIG. 1, an activated charcoal patch 100 includes a backing layer 10, an activated charcoal hydrogel 20 disposed on the backing layer and a cover layer 30 disposed on the activated charcoal hydrogel 20.

The material chosen for the backing layer 10 is one which is flexible, impermeable to the hydrogels and the active materials within the hydrogels, and, if desired, can be colored or labeled. The backing layer 10 provides support and a protective covering for the activated charcoal hydrogel 20 thereon. Suitable materials for the backing layer 10 include those known in the art for use with pressure sensitive adhesives. For example, the backing layer 10 can comprise a polyolefin, polyester, multi-layer EVA film and polyester, polyurethane or combination thereof.

The cover layer 30 is removed and discarded from the activated charcoal patch 100 to expose the activated charcoal hydrogel 20. Suitable materials for the cover layer 30 include, for example, a fluoro-silicone coated polyester or silicone coated polyester.

The activated charcoal hydrogel 20 includes activated charcoal particles dispersed within a hydrogel polymer, in which the hydrogel polymer serves as a host material for the activated charcoal particles. In addition to the activated charcoal, one or more active materials are provided in the activated charcoal hydrogel 20. As used herein, Active materials include “drug,” “pharmaceutical agent,” “pharmacologically active agent,” or any other chemical or biological material or compound suitable for transdermal administration by the methods previously known in the art and/or by the methods taught in the present invention that induces a desired biological or pharmacological effect. The effect provided by the active materials can be local, such as providing for a local anesthetic effect, or it can be systemic.

For example, the active materials may include hypnotics, sedatives, tranquilizers, anti-convulsants, muscle relaxants, analgesics, antipyretic agents, anti-inflammatory agents, local anesthetics, antispasmodics, anti-ulcer agents, anti-virals, anti-bacterials, anti-fungals, sympathomimetic agents, cardiovascular agents and antitumor agents. Non-limiting examples of active materials include nitroglycerine, scopolamine, pilocarpine, ergotamine tartrate, phenylpropanolamine, theophylline, antimicrobials tetracycline, neomycin, oxytetracycline, triclosan, sodium cefazolin, silver sulfadiazine, methylsalicylate, salicylic acid, nicotinates, methyl nicotinate, chlorhexidine gluconate, menthol, capsicum, lidocaine and benzocaine. Other drugs suitable for delivery using a transdermal system can be readily determined by persons of ordinary skill in the art. In addition, pharmacologically acceptable derivatives of the drugs, such as ethers, esters, amides, acetals, salts and the like, which are suitable for transdermal administration can be used.

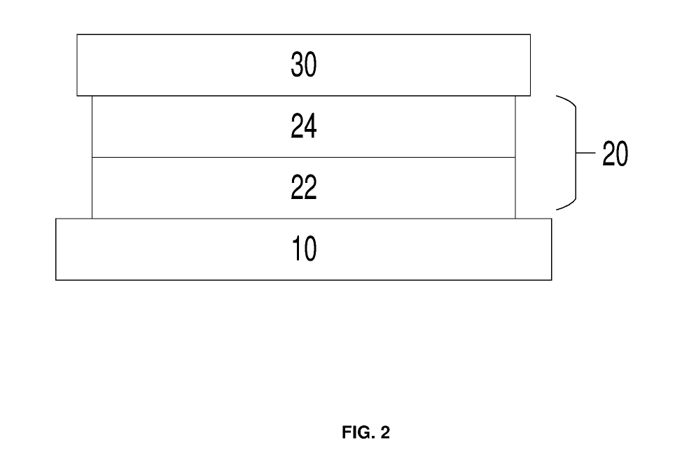

FIG. 2 is a cross-sectional view schematically illustrating hydrogel 20 of the activated charcoal patch, according to an exemplary embodiment of the activated charcoal patch. Referring to FIG. 2, the activated charcoal hydrogel 20 includes an inner hydrogel sub-layer 22 and an outer hydrogel sub-layer 24. The inner hydrogel sub-layer 22 is positioned closer to the backing layer 10, and the outer hydrogel sub-layer 24 is positioned closer to the cover layer 30. Accordingly, the outer hydrogel sub-layer 24 would be positioned closer towards the skin upon application of the activated charcoal patch 100.

By way of an example, the inner hydrogel sub-layer 22 may include an active material, while the activated charcoal particles are dispersed within the outer hydrogel sub-layer 24. Alternatively, the activated charcoal particles may be dispersed in the inner hydrogel sub-layer 22 while the active material is included in the outer hydrogel sub-layer 24. Moreover, the activated charcoal particles and an active material can be provided together within one of the inner hydrogel sub-layer 22 or the outer hydrogel sub-layer 24, and another active material can be provided in the remaining hydrogel layer.

The inner hydrogel sub-layer 22 and the outer hydrogel sub-layer 24 are both responsible for applying the active materials provided therein; the first hydrogel layer 22 is for immediate release and the second hydrogel layer 24 is for controlled release (i.e., delayed release). As such, the main polymers for the inner hydrogel sub-layer 22 and the outer hydrogel sub-layer 24 should have different swelling activation trigger characteristic so that each hydrogel layer releases its active materials under different external conditions.

In some embodiments, the compositions of the inner hydrogel sub-layer 22 and the outer hydrogel sub-layer 24 can be selected differently for the controlled release of the active materials from the inner and outer hydrogel sub-layers. That is, the inner hydrogel sub-layer 22 and the outer hydrogel sub-layer 24 can be made so that there is a significant difference in one or more other physical characteristics of the two hydrogel layers, such as the solubility, miscibility or stability of the drug or desired excipients.

For instance, the inner hydrogel sub-layer 22 and the outer hydrogel sub-layer 24 may have different thermal activation points (e.g., lower critical solution temperature, higher critical solution temperature). That is, the swelling activation trigger of the inner hydrogel sub-layer 22 differs from the swelling activation trigger of the outer hydrogel sub-layer 24. Also, the viscosity necessary for the inner hydrogel sub-layer 22 to release its active material and the viscosity necessary for outer hydrogel sub-layer 24 to release its active material may be different from each other. In any case, the activation temperature for the inner hydrogel sub-layer 22 to start swelling or to reach its targeted viscosity should be different from the activation temperature for the outer hydrogel sub-layer 24 to swell or to reach its targeted viscosity. In this way, a delay between the release of the active materials from the inner hydrogel sub-layer 22 and the release of the active materials from the outer hydrogel sub-layer 24 can be created.

In some embodiments, the inner hydrogel sub-layer 22 and the outer hydrogel sub-layer 24 may be responsive to a different pH level. In other words, release of active materials from the inner hydrogel sub-layer 22 may be triggered at a certain pH level, which is different from a pH level that triggers release of active materials from the outer hydrogel sub-layer 24.

The timing of the releasing the active materials in the inner hydrogel sub-layer 22 after the outer hydrogel sub-layer 24 may be achieved by the composition of the inner/outer hydrogel sub-layers. For example, the inner hydrogel sub-layer 22 can be formed to have a higher melting points than the outer hydrogel sub-layer 24, so that the outer hydrogel sub-layer 24 activates earlier than the inner hydrogel sub-layer 22 when the activated charcoal patch 100 is applied on the skin.

Also, the activation timing of the active materials from the inner hydrogel sub-layer 22 and the outer hydrogel sub-layer 24 may be controlled further with the relative thickness of the inner hydrogel sub-layer 22 and the outer hydrogel sub-layer 24. For example, the outer hydrogel sub-layer 24 may be formed thinner than the inner hydrogel sub-layer 22, so that the inner hydrogel sub-layer 22 can release its active materials soon after release of the active materials from the outer hydrogel sub-layer 24.

In some embodiments, one of the inner hydrogel sub-layer 22 and the outer hydrogel sub-layer 24 may be made of an acrylic-based hydrogel while the other is made of a silicone-based hydrogel. In this combination, the silicone-based hydrogel will start to release its active materials earlier than the acrylic-based hydrogel. Also, the acrylic-based hydrogel has the relatively slower delivery characteristics than the silicone-based hydrogel in terms of delivery rate of the active material therein. As such, the outer hydrogel sub-layer 24 may be formed of silicone-based hydrogel and the inner hydrogel sub-layer 22 may be formed of acrylic-based hydrogel.

In some other embodiments, one of the inner hydrogel sub-layer 22 and the outer hydrogel sub-layer 24 is formed of an acrylic-based hydrogel and the other is formed of a polyisobutylene hydrogel. In this embodiment, the acrylic-based hydrogel again has relatively slow activation and delivery characteristics than the polyisobutylene hydrogel.

If the hydrogel 20 of the activated charcoal patch 100 comprises three hydrogel sub-layers, it can comprise, for example, an acrylic-based hydrogel sub-layer sandwiched between two silicone-based hydrogel sub-layers, or a silicone-based hydrogel sub-layer in between two acrylic-based hydrogel sub-layers. Also, the hydrogel 20 of the activated charcoal patch 100 can comprise, for example, an acrylic-based hydrogel sub-layer sandwiched between two polyisobutylene hydrogel sub-layers, or a polyisobutylene hydrogel sub-layer in between two acrylic-based hydrogel sub-layers. It is also possible that each sub-layer has a different type of hydrogel, such as a silicone, polyisobutylene, and acrylate multi-layered hydrogel.

The silicone-based hydrogel can be made from silicone polymer and resin. The polymer to resin ratio can be varied to achieve different levels of tack.

The polyisobutylene hydrogel can comprise a mixture of high and low molecular weight polyisobutylenes. Specifically, high molecular weight polyisobutylenes are those with a molecular weight of at least 1,000,000. Low molecular weight polyisobutylenes are those with a molecular weight of at least 100 but less than 1,000,000. Desirably, the high molecular weight polyisobutylene comprise between about 20 and 80% by weight of the total polyisobutylene, preferably between about 40% and 50%, most preferably about 45%, and the low molecular weight polyisobutylene comprises between about 80% and 20% by weight of the total polyisobutylene, preferably between about 50% and 60%, most preferably about 55%. A specific example of a useful polyisobutylene is one which comprises 45% high molecular weight polymer (^{˜}1,250,000) and 55% low molecular weight polymer (^{˜}44,000) at approximately 25% solids in n-heptane.

The acrylic-based hydrogel can be made from various homopolymers, copolymers, terpolymers and the like of acrylic acids. They include copolymers of alkyl acrylates or methacrylates. Polyacrylates include acrylic acid, methacrylic acid, N-butyl acrylate, n-butyl methacrylate, hexyl acrylate, 2-ethylbutyl acrylate, isooctyl acrylate, 2-ethylhexyl acrylate, 2-ethylhexyl methacrylate, decyl acrylate, decylmethacrylate, dodecyl acrylate, dodecyl methacrylate, tridecyl acrylate, and tridecyl methacrylate.

Moreover, the hydrogel, and more specifically, each of the hydrogel sub-layers within the hydrogel 20 can be formed of natural polymers (e.g., xanthan gum), synthetic polymers (e.g. polyoxypropylene-polyoxyethylene block copolymer or poly-(methyl vinyl ether alt maleic anhydride)), or any combination thereof. Hydrogel polymers may be responsive to various environmental and/or external conditions, such as magnetic field, pH level changes, temperature and combination thereof. In other words, the hydrogel polymer starts to swell and allows the activated charcoals therein to serve its purpose or releases active materials contained in the hydrogel polymer. For example, magnetic field-responsive hydrogel may be Ethylene-co-vinyl acetate (EVAc). Also, non-limiting examples of pH level-responsive hydrogels include Chitosan-poly (ethylene oxide) (PEO), Poly(acrylic acid):PEO, Gelatin-PEO, Poly(acrylamide-co-maleic acid), a combination of N-vinyl pyrrolidone, polyethylene glycol diacrylate and chitosan. Non-limiting examples of thermo-responsive hydrogel include Poly(N-isopropyl acrylamide) and Poly(N-isopropyl acrylamide-co-butyl methacrylate-co-acrylic acid). It is sufficient that the activated charcoal hydrogel 20 of the activated charcoal patch 100 includes at least two or more hydrogel sub-layers that are formed of at least two different types of polymers for controlled release of the activated charcoal particles and active materials from those sub-layers.

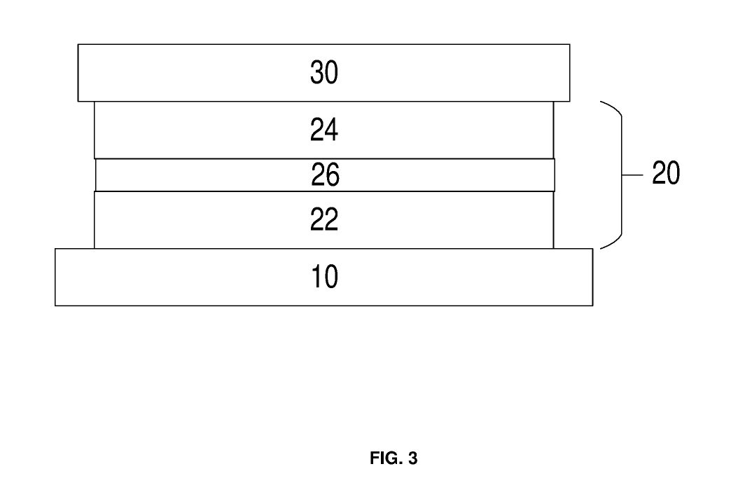

FIG. 3 is a cross-sectional view schematically illustrating the hydrogel 20, according to another exemplary embodiment of the activated charcoal patch. Referring to FIG. 3, the hydrogel 20B of the activated charcoal patch may be a multi-layered hydrogel, comprising an inner hydrogel sub-layer 22 disposed on the backing sheet 10, an outer hydrogel sub-layer 24 disposed on the inner hydrogel sub-layer 22, and an intermediate hydrogel sub-layer 26 interposed between the inner hydrogel sub-layer 22 and the outer hydrogel sub-layer 24. A removable cover layer 30 is provided on the outer hydrogel sub-layer 24.

In this configuration, the activated charcoal particles may be present in at least one of the inner hydrogel sub-layer 22, the outer hydrogel sub-layer 24 and the intermediate hydrogel sub-layer 26. Also, the inner hydrogel sub-layer 22 and the outer hydrogel sub-layer 24 may comprise one or more active materials in an amount sufficient to provide a therapeutically effective amount.

In the multi-layer hydrogel 20B, the outer hydrogel sub-layer 24 directly contacts with the skin and applies its active materials at the earliest stage of application of the activated charcoal patch 100. The inner hydrogel sub-layer 22 applies its active materials at the later stage of application of the activated charcoal patch 100. The intermediate hydrogel sub-layer 26 may serve as a delivery rate control layer for the active materials may be delivered from the inner hydrogel sub-layer 22 at a sustained rate after the early stage.

In the intermediate and later stages after application of the activated charcoal patch 100, the effect of the active materials from the outer hydrogel sub-layer 24 (e.g., delivery of the drug from the outer hydrogel sub-layer 24) decreases as the concentration of the active materials in the outer hydrogel sub-layer 24 is decreases gradually. As described above, inner hydrogel sub-layer 22 and the outer hydrogel sub-layer 24 can be made with different composition to control the activation trigger (i.e., the condition in which the inner hydrogel sub-layer 22 and the outer hydrogel sub-layer 24 start to release their active materials). However, the difference in their composition may also affect the release rate of the active materials from the hydrogel layers. It may be difficult to control the desired release rate characteristic (e.g., sustained or burst release) of the inner and outer hydrogel sub-layers due to their differences in hydrogel composition for distinct activation trigger characteristic. In other words, one of the inner and outer hydrogel sub-layers may have a faster/slower release rate for the active materials than the other. Such inconsistency in the delivery rate can be a problem, especially when the active materials of the inner hydrogel sub-layer 22 and the outer hydrogel sub-layer 24 are the same.

As such, when the inner hydrogel sub-layer 22 and the outer hydrogel sub-layer 24 comprises the same active material, the intermediate hydrogel sub-layer 26 provided in between the inner hydrogel sub-layer 22 and the outer hydrogel sub-layer 24 to create a delay in releasing the active materials from the outer hydrogel sub-layer 24 and the inner hydrogel sub-layer 22. In such cases, the intermediate hydrogel sub-layer 26 should not include the same active material as the inner hydrogel sub-layer 22 and the outer hydrogel sub-layer 24. It should be noted that the intermediate hydrogel sub-layer 26 may include active materials other than the active material of the inner and outer hydrogel sub-layers. As such, the activated charcoal may be provided in the intermediate hydrogel sub-layer 26, while the inner hydrogel sub-layer 22 and the outer hydrogel sub-layer 24 includes other active materials. Further, the intermediate hydrogel sub-layer 26 may simply be composed of polymer matrix for the hydrogel layer itself without any specific active materials.

The intermediate hydrogel sub-layer 26 interposed between the inner hydrogel sub-layer 22 and the outer hydrogel sub-layer 24 may have a third swelling activation trigger. The swelling activation trigger of the intermediate hydrogel sub-layer 26 may be the same as the swelling activation trigger of either the inner hydrogel sub-layer 22 or the outer hydrogel sub-layer 24. Alternatively, the swelling activation trigger of the intermediate hydrogel sub-layer 26 may be different from the swelling activation triggers of the inner hydrogel sub-layer 22 and the outer hydrogel sub-layer 24.

In one embodiment the intermediate hydrogel sub-layer may be formed of a pH-responsive hydrogel while the inner hydrogel sub-layer and the outer hydrogel sub-layer are formed of thermo-responsive hydrogel. Alternatively, the intermediate hydrogel sub-layer may be formed of a thermo-responsive hydrogel, while the inner hydrogel sub-layer and the outer hydrogel sub-layer are formed of pH-responsive hydrogel.

In another embodiment, all of the hydrogel sub-layers may be formed at least one of the pH-responsive hydrogel types disclosed above, but each of the hydrogel sub-layers may be configured to start swell at a distinct pH-level. Alternatively, all of the hydrogel sub-layers may be formed of at least one of thermo-responsive hydrogels mentioned above, though each of the hydrogel sub-layers has a distinct lower critical solution temperature.

In various embodiments described above, the activated charcoal particles can be provided in at least one of the inner hydrogel sub-layer 22, the outer hydrogel sub-layer 24 and the intermediate hydrogel sub-layer 26 depending on the usage of the activated charcoal particles. For instance, the outer hydrogel sub-layer 24 may be provided with activated charcoals, and the inner hydrogel sub-layer 22 may be provided with a transdermal drug. Upon application of the activated charcoal patch 100, the outer hydrogel sub-layer 24 releases the activated charcoals before the inner hydrogel sub-layer 22 releases the drug provided therein. Thus, the activated charcoals provided in the outer hydrogel sub-layer 24 is released first to absorb undesired toxins, bacteria, fungus, carcinogens and other harmful pathogens, and then the activation of the inner hydrogel sub-layer 22 follows thereafter to release the active materials that is comprised in the inner hydrogel sub-layer 22.

To serve its purpose within the aforementioned polymers of the hydrogel layers, the activated charcoal particles in at least one of the inner hydrogel sub-layer 22, the outer hydrogel sub-layer 24 and the intermediate hydrogel sub-layer 26 should have absorption index, which may be indicated by a specific surface area capacity of 500˜2000 m² per gram, porosity volume capacity of 0.2˜10 mL per gram weight, and the charge density of 0.5˜0.75 g/mL. The use of high quality activated charcoal with such properties allows excellent adsorption and absorption capacity resulting in a quicker removal of toxic chemicals and control of inflammatory processes involving the skin and surrounding tissue.

While the activated charcoal is great for extracting pathogens, toxic chemicals, inflammatory mediators and other undesired radicals from the skin, its pH level may not be suitable for direct application on the skin. For example, activated charcoal generally has pH level of about 9.26. As such, the hydrogel layer including the activated charcoal may include pH adjustment agent so that the hydrogel layer with the activated charcoal has a pH scale between 5-6.5.

In cases where certain preparation is desired on the application area prior to applying the activated charcoal, the activated charcoal can be provided in the inner hydrogel sub-layer 22. For instance, applying the activated charcoal itself would cause discomforts or cause pain at the application area. In such cases, one or more active materials for alleviating pain may be provided in the outer hydrogel sub-layer 24 while the activated charcoal is provided in the inner hydrogel sub-layer 22. In this way, the pain relieving active materials of the outer hydrogel sub-layer 24 is released first to anaesthetize the application area, then the inner hydrogel sub-layer 22 is activated after the outer hydrogel sub-layer 24 to release the activated charcoals on the application area to absorb undesired toxins, bacteria, fungus, carcinogens and other harmful pathogens. It should be appreciated that the outer hydrogel sub-layer 24 may comprise both the activated charcoals particles as well as the pain relief active materials so that the activated charcoal particles and the pain relief active materials in the outer hydrogel sub-layer 24 is released prior to the release of the active materials in the inner hydrogel sub-layer 22.

Also included within the scope of the invention are bioactive agents, such as insect repellants, sun screens, cosmetic agents, etc. Accordingly, the outer hydrogel sub-layer 24 may include an active material to temporarily widen pores at the skin area where the activated charcoal patch 100 is applied, for more effective extraction of pathogens, toxic chemicals, inflammatory mediators by the activated charcoal. Extracting the pathogens, toxic chemicals, inflammatory mediators and other undesired radicals from the skin can increase the absorption rate of the active materials of the inner hydrogel sub-layer 22 by the skin.

The active materials provided in the inner hydrogel sub-layer 22 may include a moisturizing agent. Non-limiting examples of the moisturizing agent include glycerin, sorbitol, propylene glycol, ethylene glycol, diethlyene glycol, polyethylene glycol, polypropylene glycol, 1,3-butylene glycol, hyaluronic acid. Hyaluronic acid has the effects against skin photo-damage and aging. Also it gives rise to increase of tissue hydration. Therefore, it is common ingredient in skin care products. Effect of the moisturizing agents in the inner hydrogel sub-layer 22 can be highly increased by the activated charcoals provided in the outer hydrogel sub-layer 24.

Moreover, the active materials in the inner hydrogel sub-layer 22 may include essential oils. Non-limiting examples of the essential oils, which may be provided in the inner hydrogel sub-layer 22 include avocado oil, kale oil, menthol, arbutin, olive oil, herbal extracts, flaxseed power/oil, eucalyptus oil. The essential oils provided in the inner hydrogel sub-layer 22 can help opening the hair follicle of the skin and support on receiving the nutrients.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. 1. An activated charcoal patch, comprising:
a backing sheet;
a cover sheet;
an outer hydrogel sub-layer having a first active material; and
an inner hydrogel sub-layer having a second active material,
wherein activated charcoal is dispersed within at least one of the inner hydrogel sub-layer and the outer hydrogel sub-layer.

2. The activated charcoal patch of claim 1, wherein the outer hydrogel sub-layer has a first swelling activation trigger, and the inner hydrogel sub-layer has a second swelling activation trigger.

3. The activated charcoal patch of claim 2, wherein the outer hydrogel sub-layer and the inner hydrogel sub-layer have a first lower critical solution temperature and a second lower critical solution temperature, respectively.

4. The activated charcoal patch of claim 2, wherein the first swelling activation trigger is pH change of the inner hydrogel sub-layer across a predetermined threshold pH, and wherein the second swelling activation trigger is temperature change of the outer hydrogel sub-layer across a predetermined threshold temperature.

5. The activated charcoal patch of claim 2, wherein the first swelling activation trigger is temperature change of the outer hydrogel sub-layer across a predetermined threshold temperature, and wherein the second swelling activation trigger is pH change of the inner hydrogel sub-layer across a predetermined threshold pH.

6. The activated charcoal patch of claim 2, wherein each of the outer hydrogel sub-layer and the inner hydrogel sub-layer is formed of at least one of an acrylic-based hydrogel, a polyisobutylene hydrogel and a silicone-based hydrogel.

7. The activated charcoal patch of claim 1, wherein the first active material and the second active material are selected from the group consisting of hypnotics, sedatives, tranquilizers, anti-convulsants, muscle relaxants, analgesics, antipyretic agents, anti-inflammatory agents, local anesthetics, antispasmodics, anti-ulcer agents, anti-virals, anti-bacterials, anti-fungals, sympathomimetic agents, cardiovascular agents and antitumor agents.

8. The activated charcoal patch of claim 7, wherein the first active material and the second active material are selected from the group consisting of nitroglycerine, scopolamine, pilocarpine, ergotamine tartrate, phenylpropanolamine, theophylline, antimicrobials tetracycline, neomycin, oxytetracycline, triclosan, sodium cefazolin, silver sulfadiazine, methylsalicylate, salicylic acid, nicotinates, methyl nicotinate, chlorhexidine gluconate, menthol, capsicum, lidocaine and benzocaine.

9. The activated charcoal patch of claim 1, wherein one of the outer hydrogel sub-layer and the inner hydrogel sub-layer is a thermos-responsive hydrogel layer and the other one of outer hydrogel sub-layer and the inner hydrogel sub-layer is a pH-responsive hydrogel layer.

10. The activated charcoal patch of claim 1, further comprising an intermediate hydrogel sub-layer interposed between the inner hydrogel sub-layer and the outer hydrogel sub-layer.

11. The activated charcoal patch of claim 10, wherein the intermediate hydrogel sub-layer has a third swelling activation trigger, which is different from the first swelling activation trigger and the second swelling activation trigger.

12. The activated charcoal patch of claim 10, wherein the intermediate hydrogel sub-layer has a third swelling activation trigger, which is same as at least one of the first swelling activation trigger and the second swelling activation trigger.

13. The activated charcoal patch of claim 10, wherein the intermediate hydrogel sub-layer is formed of a pH-responsive hydrogel, and wherein the inner hydrogel sub-layer and the outer hydrogel sub-layer are formed of thermo-responsive hydrogel.

14. The activated charcoal patch of claim 10, wherein the intermediate hydrogel sub-layer is formed of a thermo-responsive hydrogel, and wherein the inner hydrogel sub-layer and the outer hydrogel sub-layer are formed of pH-responsive hydrogel.

15. The activated charcoal patch of claim 10, wherein all of the hydrogel sub-layers are formed of at least one of thermo-responsive hydrogels, and wherein each of the hydrogel sub-layers has a distinct lower critical solution temperature.

16. The activated charcoal patch of claim 10, wherein all of the hydrogel sub-layers are formed of at least one of pH-responsive hydrogels, and wherein each of the hydrogel sub-layers starts to swell at a distinct pH-level.
